(19) United States
(12) Patent Application Publication
(10) Pub. No.: US 2020/0222477 A1
(43) Pub. Date: Jul. 16, 2020

(54) Method to Curtail the Progression of Non-Muscle Invasive Bladder Cancer To Muscle-Invasive Cancer

(71) Applicant: Trizell Ltd., Chinnor (GB)

(72) Inventors: Nigel PARKER, Chinnor (GB); Seppo YLA-HERTTUALA, Kuopio (FI); David Sawutz, Toronto (CA)

(73) Assignee: Trizell Ltd., Chinnor (GB)

(21) Appl. No.: 16/702,782

(22) Filed: Dec. 4, 2019

Related U.S. Application Data
(60) Provisional application No. 62/790,524, filed on Jan. 10, 2019.

(51) Int. Cl. A61K 35/761; A61P 35/00; A61K 9/00

(52) U.S. Cl. CPC A61K 35/761; A61K 9/0034; A61P 35/00

(57) ABSTRACT

Viral gene therapy provides a successful treatment for high-grade, non-muscle invasive bladder cancer resistant to, or recurrent after, treatment with intravesical bacillus Calmette-Guérin (BCG) vaccine. Our therapy provides the first and only way to reliably curtail the progression of superficial, non-muscle invasive cancer into more lethal muscle-invasive cancer.

### RELATED APPLICATIONS

This application claims priority to U.S. provisional patent filing Ser. No. 62/790,542 filed 10 Jan. 2019, the contents of which are here incorporated by reference.

### UNITED STATES GOVERNMENT INTEREST

None.

### THE NAMES OF THE PARTIES TO A JOINT RESEARCH AGREEMENT

None.

### REFERENCE TO A SEQUENCE LISTING

None.

### STATEMENT REGARDING PRIOR DISCLOSURES BY THE INVENTOR OR A JOINT INVENTOR

None.

### BACKGROUND

We have found a way to dramatically reduce the progression of superficial, non-lethal bladder cancer to metastatic, lethal cancer.

Bladder cancer currently has a prevalence of ≈700,000 cases in the USA. Each year, we see >80,000 newly-diagnosed cases and 17,000 deaths. From diagnosis to death, bladder cancer is one of the most expensive cancers to treat.

Approximately 70% of all newly diagnosed bladder tumors are superficial, or “non-muscle invasive,” bladder cancers (NMIBC). While bladder cancer may not be particularly dangerous as long as it remains non-muscle invasive, when it progresses to muscle invasive it is quite dangerous.

NMIBCs may be classified according to the extent and type of growth, classifications providing a continuum of risk. Carcinoma in situ (CIS) and stage Ta pose less of a risk of metastasis. Stage T1 is greater risk, and stage T1 high-grade (T1Hg) bladder cancer is at the aggressive end of the spectrum.

T1 bladder cancer represents 5% to 20% of NMIBC and is defined as an invasion into the lamina propria without invasion into the muscularis propria. World Health Organization pathology guidelines (2004) recommend a re-naming or change from the previously-used classification of grades of G1, G2 or G3 to that of low- or high-grade papillary urothelial carcinoma. Pathology reports should identify whether muscle tissue is present in the resected specimen. One study described that a pathology report of a repeat resection of T1 disease found the incidence of “understaging” (underestimating the severity of the cancer) was only 14% when muscle tissue was present compared to 49% when muscle tissue was absent in the initial specimen. It is recommended that pathologists report the extensiveness of T1 disease since some studies have reported that focal lamina propria invasion may present fewer risks than extensive involvement. While under-staging remains problematic, over-staging of T1 disease in pathology reports has also been described; about 25% to 35% of cases were found to be stage Ta disease when reviewed by a second pathologist. This is significant to the discussion of cystectomy for treatment of stage T1 disease.

Optimal management of T1Hg bladder cancer begins with complete initial transurethral resection of the bladder tumor, a procedure referred to as “TURBT.” A rectal or bimanual exam under anesthesia is recommended on presentation of TURBT to evaluate any local extension of the tumor. Following initial TURBT, attempts should be made to provide complete tumor resection including muscle in the specimen. To avoid inadvertent perforation of the bladder and the concomitant risk of infection, a surgeon must be specially trained to follow the interior contour of the bladder when excising tumor tissue. Anesthetic paralysis or an obturator nerve block agent while resecting an area near the obturator nerve may be helpful in preventing adductor contraction and potential bladder perforation.

Sending separate pathology specimens from the superficial tumor and deeper samples or “bites” of the muscle tissue may assist pathologists in accurately staging the depth of invasion. An immediate postoperative dose of intravesical chemotherapy (mitomycin 40 mg in 40 cc water) has been reported to decrease the frequency of recurrence.

Under-staging is significant in patients with a recent diagnosis of T1Hg bladder cancer. The standard of care has progressed to mandatory second resection (restaging TURBT) in cases of T1Hg bladder cancer. The recommended time-frame for repeat resection is about 4 weeks for patients with T1Hg bladder cancer. At time of re-resection, 45% to 76% of patients demonstrate residual bladder cancer and 29% to 40% are upstaged to muscle-invasive disease.

Concomitant carcinoma in situ and/or multifocality are known negative prognostic factors for recurrence and progression. Reports of clinical under staging in up to 40% of patients with stage T1 bladder tumors after primary resection also led to a cautious therapeutic approach to primary stage T1 bladder tumors.

The prior art says that the ideal treatment for primary stage T1 grade 3 bladder cancer remains controversial. The main therapeutic options after initial transurethral resection are observation, repeat resection, cystectomy (removal of the bladder) and intravesical therapy. Intravesical therapy includes two currently-approved agents: bacillus Calmette-Guérin (BCG) vaccine and valrubicin.

Bacillus Calmette-Guérin (BCG) vaccine is a live (infective) attenuated bacillus. It is primarily used against tuberculosis (TB). It is also used as second-line therapy in bladder cancer, after tumor resection. Compared to resection alone, the prior art teaches that BCG slightly reduces the risk of cancer recurrence and cancer progression to a metastatic or muscle-invasive form.

For example, Shahin, Osama et al., A Retrospective Analysis Of 153 Patients Treated With Or Without Intravesical Bacillus Calmette-Guérin For Primary Stage T1 Grade 3 Bladder Cancer: Recurrence, Progression And Survival, 169 J. Urology 96 (2003) teaches that after transurethral bladder resection alone, the recurrence rate for stage T1 grade 3 (or high grade) bladder tumors is 69% to 80%, and the progression rate is 33% to 48%. See pg. 96 col. 1. In his own set of patients, disease recurred in 75% of the patients treated with resection alone, and in only 70% of the patients treated with transurethral resection plus BCG. Shahin thus teaches that BCG reduces the risk of cancer recurrence, but reduces it only slightly—from 75% to 70%.

Shahin similarly teaches that tumor progressed in 36% of the patients treated with resection alone, but in only 33% of patients treated with transurethral resection plus BCG. Shahin thus teaches that BCG reduces the risk of cancer progression, but reduces it only slightly—from 36% to 33%. Shahin thus concludes that even with BCG as second-line therapy, in two-thirds of patients disease progresses and ultimately requires complete bladder removal (cystectomy) or is fatal:
“Our results suggest that intravesical BCG therapy after transurethral bladder resection for stage T1 grade 3 bladder cancer may delay the time to recurrence and cystectomy but it does not substantially alter the final outcome. Our findings reflect the rule of 30% for stage T1 grade 3 cancer, namely approximately 30% of patients never have recurrence, 30% ultimately die of metastatic disease and 30% require deferred cystectomy.”

### Id.

Live BCG vaccine is intended to stimulate an anti-cancer immune response. BCG, however, fails in 70% of patients. The art teaches that when BCG therapy fails, then the patient's immune system is likely weak or impaired, and thus unable to produce an immune response that is adequately strong to combat cancer. The art thus teaches that if BCG therapy fails, to try an alternative, non-immune approach, and administer a standard cytotoxic chemotherapy drug, valrubicin.

The efficacy of valrubicin third-line salvage therapy is taught by Steinberg, G. et al., Efficacy and Safety of Valrubicin for the Treatment of Bacillus Calmette-Guérin Refractory Carcinoma in Situ of the Bladder, 163 J. Urology 761 (2000). Steinberg (2000) assessed the efficacy and safety of intravesical valrubicin for the treatment of carcinoma in situ in patients with failure or recurrence after bacillus Calmette-Guérin (BCG) and who otherwise would have undergone cystectomy. A total of 90 patients with recurrent carcinoma in situ after failed multiple prior courses of intravesical therapy, including at least 1 course of BCG, participated in his open label, non-comparative study. Each patient received 6 weekly instillations of 800 mg. intravesical valrubicin. Disease evaluations were made at baseline and 3-month intervals following treatment. Evaluations included cystoscopy with biopsy and urine cytology. Toxicity was noted throughout treatment and follow-up.

Steinberg (2000) finds that of 90 patients, at the last evaluation only 7 (8%) remained disease-free. Steinberg finds recurrence in 79 (88%) patients, of which 44 (49% of the total patient population) had recurrent disease or progression so severe as to require radical cystectomy. Of the 46 patients who did not undergo complete cystectomy, four (9%) died of (apparently metastatic) bladder cancer.

Salvage therapy of last resort is a radical cystectomy. It is a major surgical operation because it is a complicated procedure to remove the bladder and pelvic organs, lymph nodes with intestinal reconstruction of the urinary tract. It requires 6 to 8 hours under anaesthesia, 5 to 10 days in hospital, and a 6 to 8 week recovery period. There is a 90-day mortality rate of 1% for patients <70 years of age to 9% for patients 80 years of age and older. Up to 50% of patients experience significant post-operative complications. Cystectomy also imposes a significant impact on quality of life and body image. Thus, while metastatic disease is often fatal, many patients nonetheless steadfastly refuse to consider radical cystectomy due to concerns about quality of life, urinary continence or ostomy, and sexual dysfunction.

The art thus teaches that in patients with stage T1 high grade (f/k/a grade 3) bladder cancer treated with resection alone, disease recurs in 75% and progresses in 36% of patients. See Shahin (2003). In patients treated with resection and BCG, disease recurs in 70% and progresses in 33% of patients. See id. Treating such recurrent patients with intravesical valrubicin improves matters but slightly: only 8% remain disease-free, and fully half (49%) require radical cystectomy.

There thus remains a need for therapy that prevents recurrence and progression of NMIBC.

### BRIEF SUMMARY

The art teaches that BCG therapy fails in 70% of patients. BCG failure implies that the patient's immune system is weak or impaired. The art thus teaches to treat BCG-failed patients with valrubicin, a cytotoxic agent that kills dividing cells, regardless of whether they are cancerous or healthy. The art, however, still teaches that this fails to prevent recurrence and progression in the majority of patients.

We tested an alternative approach. Rather than respond to BCG failure by administering a cytotoxic that kills dividing cells, we tested an approach to repair the patient's (ostensibly weak or impaired) immune system. Our approach entailed administering a viral gene therapy vector with a transgene that expresses interferon, an immune-modulating cytokine. We tested our approach in the most difficult-to-treat patients, i.e., patients with high grade disease that remained resistant to or recurrent after resection and BCG therapy.

We found—surprisingly—that our approach dramatically reduces recurrence and almost entirely eliminates progression. Our success was surprising because the art teaches that BCG-resistant or -recurrent patients have such weakened immune systems that even non-immune therapy (e.g., valrubicin) is not particularly helpful.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the result of radical cystectomy with external ostomy bag.

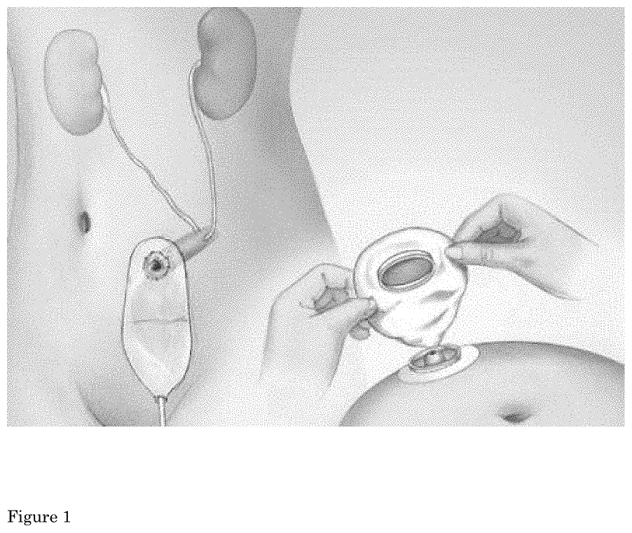

FIG. 2 is a flow-chart of the overall study design and data-analysis plan.

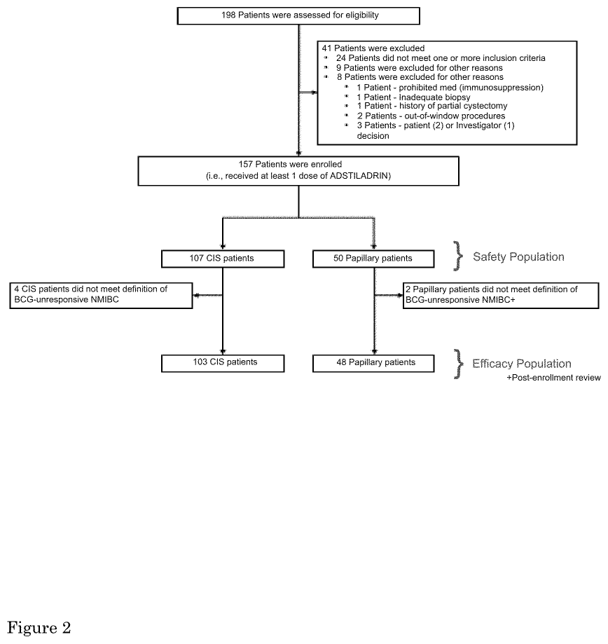

FIG. 3 is a flow-chart of the study design and data-analysis plan for patients diagnosed CIS at study initiation.

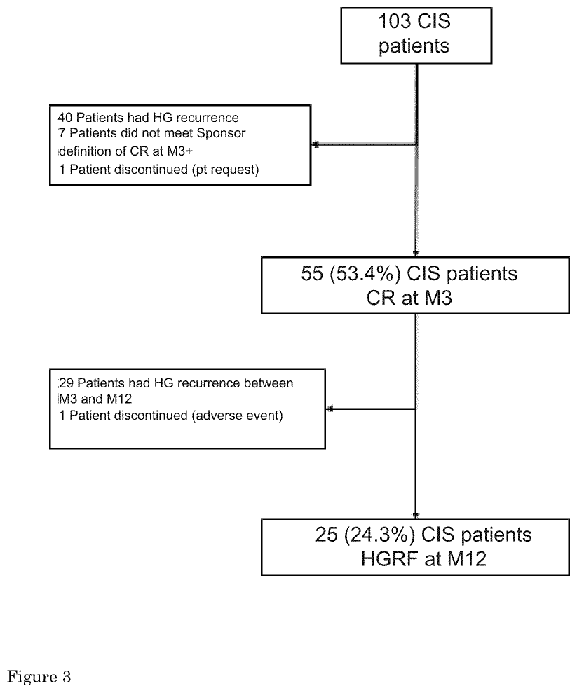

FIG. 4 is a flow-chart of the study design and data-analysis plan for patients diagnosed papillary-only at study initiation.

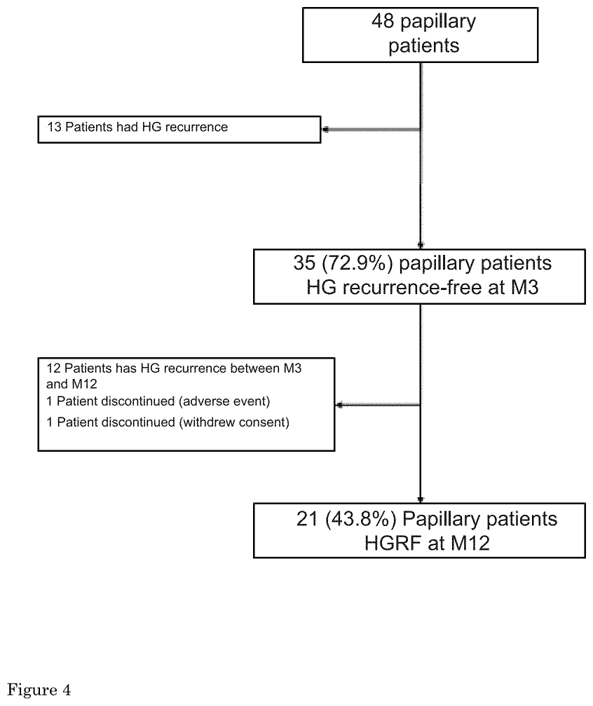

FIG. 5 is a Kaplan-Meyer plot of durability of complete response in patients with CIS who achieved CR at Month 3, Durability of Complete Response in Patients with CIS (2). The plotted population includes only patients who achieved a CR at t=3 months; thus, t=0 on the plot is three months after first instillation. The month 12 follow-up visit window opens at t=9 months on the plot.

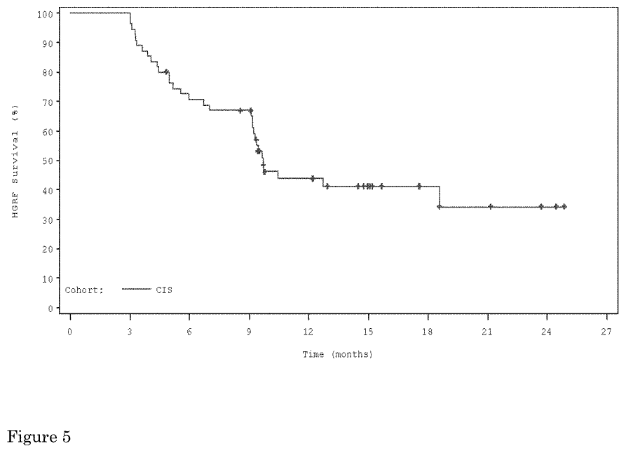

FIG. 6 is a Kaplan-Meyer plot of durability of complete response in patients with papillary cancer who achieved CR at Month 3, Durability of Complete Response in Patients with papillary cancer. The plotted population includes all papillary patients; thus, t=0 on the plot is zero (0) months after first instillation. The month 12 follow-up visit window opens at t=12 months on the plot.

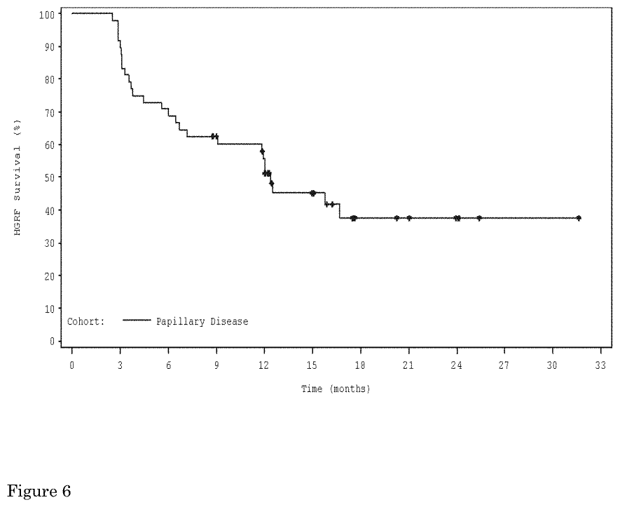

### DETAILED DESCRIPTION

Materials & Methods:

Viral Gene Therapy Vector.

Nadofaragene firadenovec is a recombinant, replicatin-deficient adenovirus bearing a transgene for human interferon alpha 2b. It is often called “rAd-IFN.” The virus delivers the human IFNα2b gene, resulting in IFNα2b protein expression. Viral delivery of the interferon gene provides an increased level and duration of exposure of the bladder wall and tumor tissues to the IFNα2b protein. This largely overcomes the therapeutic limitations the art has encountered with intravesical administration of INTRON® A IFNα2b polypeptide.

IFNα2b has pleiotropic effects against cancer growth. It has anti-tumor activity by direct TRAIL-mediated cell death. It also inhibits angiogenesis. Further, it sstimulates both the innate and the adaptive immune systems.

We have performed vector shedding studies that indicate that intravesical instillation of rAd-IFN produces a very low likelihood of systemic exposure to rAd-IFN-derived DNA. (data not shown).

Phase 3 Trial Study Design:

We believe that single-arm trials are appropriate for assessment of therapies for patients with BCG-unresponsive disease (CIS with or without resected papillary disease) because currently no effective medical therapies are available, and the only alternative is radical cystectomy. Thus, we believe that patients with carcinoma in situ (CIS) at trial entry can be studied in either a randomized, controlled trial or a single-arm trial. We believe that in BCG-unresponsive NMIBC, a single-arm clinical trial with complete response rate and duration of response as the primary endpoint can provide primary evidence of effectiveness to support a marketing application. Our study design thus can include patients with completely resected lesions and no evidence of CIS in these single-arm trials but does not include them in the evaluation of the primary efficacy endpoint. However, we include these patients in the safety analysis.

We finalized our design of our Phase 3 study following consultation with both the United States Food & Drug Administration (at a confidential “end of Phase 2” meeting) and The Society of Urologic Oncology Clinical Trials Consortium (SUO CTC). We used a patient population representative of that proposed for commercial use. At our Initial Comprehensive Multidisciplinary BTD meeting, we agreed that the primary endpoint was a “complete response” (or “CR”) at any time in patients with CIS±concomitant high-grade Ta/T1 disease. The durability of CR over time was a key secondary endpoint. We decided to not pool the Phase 2 safety data and the Phase 3 safety data because that was not necessary. We planned to collect interim results after nine months of treatment, and collect final results after twelve months of treatment.

We thus planned a single, Phase 3, open-label efficacy and safety registration study in patients with BCG-unresponsive NMIBC (rAd-IFN-CS-003) conducted in the USA. Our study plan encompassed 107 patients with CIS±concomitant high-grade Ta/T1 disease (“CIS”), and 50 patients with high-grade Ta/T1 disease (“papillary disease”). The primary objective was to evaluate the complete response rate in patients with CIS (with or without concomitant high-grade Ta or T1 papillary disease). The primary endpoint was whether a patient with CIS (with or without concomitant high-grade Ta or T1 papillary disease) responds to treatment, defined as complete response at any time after first administration of ADSTILADRIN® brand nadofaragene firadenovec (2.25×10¹³ virus particles (vp) in a total volume of 75 mL) with Syn3® brand (3α,5β,7α,12α)-N-[3-[(4-O-D-galactopyranosyl-D-glucanoyl)amino]propyl]-3,7,12-trihydroxy-N-[3-[[(3α,5β,7α,12α)-3,7,12-trihydroxy-24-oxocholan-24-yl]amino]propyl]-cholan-24-amide (generically referred to as “NODA”), a surfactant, administered by intravesical instillation every three months. Our goals were to evaluate the durability of complete response in patients with CIS (with or without concomitant high-grade Ta or T1 papillary disease) who achieve a complete response; to evaluate the rate of event-free survival, where event-free survival is defined as high-grade recurrence free survival in patients with high-grade Ta or T1 papillary disease (without concomitant CIS); and to evaluate the durability of event-free survival in patients with high-grade Ta or T1 papillary disease (without concomitant CIS), who have no recurrence of high-grade Ta or T1 papillary disease. For comparison purposes, this will also be evaluated in patients with CIS.

A secondary endpoint included durability of complete response in patients with CIS (with or without concomitant high-grade Ta or T1 papillary disease) who achieve a complete response. Another was whether or not a patient with high-grade Ta or T1 papillary disease (without concomitant CIS) responds to treatment, defined as absence of recurrence of high-grade Ta or T1 papillary disease. Another was the durability of event-free survival in patients with high-grade Ta or T1 papillary disease (without concomitant CIS) who have no recurrence of high-grade Ta or T1 papillary disease.

To handle missing data, we decided that where a patient misses a visit and a HG recurrence is not observed at the next visit, the patient is considered HG recurrence-free at the missed visit. Where a patient misses a visit and HG recurrence is observed at the next visit, the outcome of recurrence is “back-dated” to the previous visit. Where a patient drops out of treatment and did not have a response assessment at a visit, this is considered a HG recurrence at that visit. Thus, the actual or proxy outcomes for all patients in the Month 12 analysis would be known.

Patient Disposition statistics are shown on the following table:

Phase 3 Trial Patient Disposition

| | CIS | | Papillary | | Total | |
|---|---|---|---|---|---|---|
| | n | (%) | n | (%) | n | (%) |
| Patients Enrolled (Received | 107 | 100 | 50 | 100 | 157 | 100 |
| First Dose) (1) | | | | | | |

Patients Treated

| | | | | | | |
|---|---|---|---|---|---|---|
| At 3 months | 63 | 58.9 | 35 | 70.0 | 98 | 62.4 |
| At 6 months | 43 | 40.2 | 30 | 60.0 | 73 | 46.5 |

Analysis Populations

| | | | | | | |
|---|---|---|---|---|---|---|
| Efficacy Analysis Set | 103 | 96.3 | 48 | 96.0 | 151 | 96.2 |
| Safety Analysis Set | 107 | 100 | 50 | 100 | 157 | 100 |

Note:
CIS = CIS Only, Ta + CIS, T1 + CIS; Papillary Disease - Ta & T1.
Percentage is calculated using the total number of treated patients as the denominator.
(1) Patients enrolled include all patients who signed the informed consent form, were enrolled and received the first dose.

Patient demographic data is shown on the Table below:

Phase 3 Trial Demographics and Baseline Characteristics

| | | | |
|---|---|---|---|
| Demographic/Baseline | | Papillary | |
| Characteristics | CIS | Disease | Total |
| Statistic/Category | (n = 107) | (n = 50) | (n = 157) |

Age at Informed Consent (years)

| n | 107 | | 50 | | 157 | |
|---|---|---|---|---|---|---|
| Mean | 71.2 | | 70.1 | | 70.8 | |
| Standard Deviation | 8.88 | | 9.78 | | 9.16 | |
| Median | 72.0 | | 71.0 | | 71.0 | |
| Q1, Q3 | 66.0, | 77.0 | 64.0, | 78.0 | 66.0, | 77.0 |
| Minimum, Maximum | 44, | 89 | 39, | 87 | 39, | 89 |

Age Group at Informed Consent (n, %)

| | | | | | | |
|---|---|---|---|---|---|---|
| <65 Years | 25 | (23.4) | 13 | (26.0) | 38 | (24.2) |
| >=65 Years | 82 | (76.6) | 37 | (74.0) | 119 | (75.8) |

Sex (n, %)

| | | | | | | |
|---|---|---|---|---|---|---|
| Male | 95 | (88.8) | 34 | (68.0) | 129 | (82.2) |
| Female | 12 | (11.2) | 16 | (32.0) | 28 | (17.8) |

Race (n, %)

| | | | | | | |
|---|---|---|---|---|---|---|
| White | 99 | (92.5) | 47 | (94.0) | 146 | (93.0) |
| Black or African American | 6 | (5.6) | 2 | (4.0) | 8 | (5.1) |
| American Indian or Alaska Native | 0 | (0.0) | 0 | (0.0) | 0 | (0.0) |
| Native Hawaiian or Pacific Islander | 0 | (0.0) | 0 | (0.0) | 0 | (0.0) |
| Asian | 2 | (1.9) | 1 | (2.0) | 3 | (1.9) |
| Other | 0 | (0.0) | 0 | (0.0) | 0 | (0.0) |
| Multiple | 0 | (0.0) | 0 | (0.0) | 0 | (0.0) |

Ethnicity (n, %)

| | | | | | | |
|---|---|---|---|---|---|---|
| Hispanic or Latino | 3 | (2.8) | 1 | (2.0) | 4 | (2.5) |
| Not Hispanic or Latino | 99 | (92.5) | 49 | (98.0) | 148 | (94.3) |
| Not Reported | 1 | (0.9) | 0 | (0.0) | 1 | (0.6) |
| Unknown | 4 | (3.7) | 0 | (0.0) | 4 | (2.5) |

Height (cm)

| n | 107 | | 49 | | 156 | |
|---|---|---|---|---|---|---|
| Mean | 174.7 | | 170.9 | | 173.5 | |
| Standard Deviation | 9.89 | | 10.37 | | 10.16 | |
| Median | 176.0 | | 172.0 | | 175.0 | |
| Q1, Q3 | 170.0, | 180.0 | 167.0, | 178.0 | 168.0, | 180.0 |
| Minimum, Maximum | 152, | 201 | 150, | 198 | 150, | 201 |

Baseline Weight (kg)

| n | 107 | | 50 | | 157 | |
|---|---|---|---|---|---|---|
| Mean | 90.14 | | 85.88 | | 88.78 | |
| Standard Deviation | 20.919 | | 18.639 | | 20.259 | |
| Median | 88.20 | | 83.95 | | 85.90 | |
| Q1, Q3 | 77.10, | 100.90 | 74.40, | 99.10 | 75.30, | 99.80 |
| Minimum, Maximum | 51.4, | 150.6 | 45.4, | 133.8 | 45.4, | 150.6 |

Baseline Body Mass Index (kg/m\hat{ }2)

| n | 107 | | 49 | | 156 | |
|---|---|---|---|---|---|---|
| Mean | 29.38 | | 29.07 | | 29.28 | |
| Standard Deviation | 5.716 | | 4.952 | | 5.473 | |
| Median | 27.85 | | 28.66 | | 28.28 | |
| Q1, Q3 | 25.18, | 32.95 | 26.17, | 32.07 | 25.19, | 32.33 |
| Minimum, Maximum | 20.3, | 46.4 | 18.9, | 39.8 | 18.9, | 46.4 |

Baseline ECOG Status (n, %)

| | | | | | | |
|---|---|---|---|---|---|---|
| 0 | 97 | (90.7) | 43 | (86.0) | 140 | (89.2) |
| 1 | 7 | (6.5) | 6 | (12.0) | 13 | (8.3) |
| 2 | 3 | (2.8) | 1 | (2.0) | 4 | (2.5) |

Human Phase 3 Clinical Trial Results

Data Analysis Sets

| Analysis | | |
|---|---|---|
| Set | N | Definition |
| Safety | 157 | All patients who received at least 1 dose of |
| | | ADSTILADRIN |
| | | Used for all safety analyses |
| Efficacy | 151 | All patients in the SAS with diagnosis of high-grade, |
| | | BCG unresponsive NMIBC |
| | | Used as primary analysis set for analyses of efficacy |
| | | data |
| Per | 147 | All patients in the EAS Set who had no major protocol |
| Protocol | | violation and either: |
| | | Completed Month 12 Efficacy Assessment Visit at |
| | | earliest Day + 357 and at latest Day + 396; OR |
| | | Withdrew before Month 12 Efficacy Assessment |
| | | Visit because of disease recurrence or progression, |
| | | death, an adverse event related to the disease or |
| | | treatment, or lack of tolerability |

Incidence of Complete Response at Any Time Patients with CIS

| Patients Who Have Achieved a | | |
|---|---|---|
| Complete Response (n, %) | CIS (N = 103) | |
| By 3 months | 55 | (53.4) |
| During months 4 to 6 | 0 | (0.0) |
| During months 7 to 9 | 0 | (0.0) |
| During months 10 to 12 | 0 | (0.0) |
| Total | 55 | (53.4) |

Incidence of HGRF Survival Patients with
CIS That Achieved a Complete Response

| | |
|---|---|
| Patients who have achieved | |
| HGRF survival at: (n, %) | CIS (N = 55) |

| | | | |
|---|---|---|---|
| 3 | months | 55 | (100.0) |
| 6 | months | 42 | (76.4) |
| 9 | months | 36 | (65.5) |
| 12 | months | 25 | (45.5) |

Durability of Complete Response Patients with CIS (N = 103)

| Patients who achieved a CR | 55 | |
|---|---|---|
| Patients with treatment failure after CR (n, %) | | |
| High-grade recurrence without muscle invasive | 30 | (54.5) |
| disease progression | | |
| High-grade recurrence with muscle invasive | 1 | (1.8) |
| disease progression | | |
| Death | 0 | (0.0) |
| Patients censored (n, %) | 24/55 | (43.6) |
| K-M estimate of durability of CR, (mo.): | | |
| Median | 9.69 | |
| 95% CI for median | 9.17, | NE |

Papillary Cohort:

Incidence of High Grade Recurrence Free
Survival Papillary Cohort (n = 48)

Patients achieving
HGRF survival (n, %)

| | | |
|---|---|---|
| 3 | months | 35 (72.9) |
| 6 | months | 30 (62.5) |
| 9 | months | 28 (58.3) |
| 12 | months | 21 (43.8) |

Durability of HGRF Survival Papillary (N = 48)

| Patients with recurrence of high-grade disease (n, %) | 27 | (56.3) |
|---|---|---|
| Patients with treatment failure after CR (n, %) | | |
| High-grade recurrence w/o muscle invasive | 25 | (52.1) |
| disease progression | | |
| High-grade recurrence with muscle invasive | 1 | (2.1) |
| disease progression | | |
| Death | 1 | (2.1) |
| Patients censored (n, %) | 21 | (43.8) |
| K-M estimate of durability of CR, (mo.): | | |
| Median | 12.35 | |
| 95% CI for median | 6.67, | NE |

Reviewing our combined population safety data, our data show that no patient had an adverse event leading to death. Six patient deaths were reported, but each patient who died had already been withdrawn from study treatment and was merely in the long term follow-up period. Further, each of the six deaths occurred at least four (4) months after the last administration of the study drug.

| Patient ID | Cause of Death |
|---|---|
| 100-001 | Cardio-respiratory collapse |
| 118-002 | Pulse-less electrical activity (PEA) arrest |
| 122-001 | Unknown |
| 126-003 | Cardiac arrest |
| 104-004* | Metastatic lung cancer |
| 105-002 | Pneumonia |

Our data thus show that we have successfully found a treatment of high-grade, BCG-unresponsive non-muscle invasive bladder cancer. As importantly, we found the first and only way to reliably curtail the progression of superficial (non-muscle invasive) cancer into more lethal muscle-invasive cancer.

We claim:

1. A method for preventing the progression of non-muscle invasive bladder cancer to muscle-invasive cancer, comprising instilling a virus intra-vesically into the bladder of a human that has been diagnosed with non-muscle invasive bladder cancer, said virus instilled in an amount effective to prevent the progression of non-muscle invasive bladder cancer to muscle-invasive cancer.

2. The method of claim 1, where the virus does not replicate in normal healthy bladder cells.

3. The method of claim 1, where the virus contains a human interferon gene.

4. The method of claim 1, where the human has been diagnosed with high-grade, non-muscle invasive bladder cancer.

5. The method of claim 1, where the human has been diagnosed with non-muscle invasive bladder cancer resistant to or recurrent after treatment with bacillus Calmette Guérin.

6. The method of claim 1, further comprising instilling a surfactant intra-vesically into the bladder.

7. The method of claim 1, where the human has been diagnosed with non-muscle invasive bladder cancer recurrent after resection.

8. A method for treating high-grade, bacillus Calmette Guérin-unresponsive non-muscle invasive bladder cancer in a human, comprising administering by intravesical instillation, about once every three months, to a human that has been diagnosed with high-grade bacillus Calmette Guérin-unresponsive non-muscle invasive bladder cancer, NODA and about 2.25×10¹³ virus particles (vp) of nadofaragene firadenovec.
